(19) United States
(12) Patent Application Publication
(10) Pub. No.: US 2026/0093784 A1
(43) Pub. Date: Apr. 2, 2026

(54) TRAINING DEVICE, TRAINING METHOD, ATTRIBUTE DATA GENERATION DEVICE, ATTRIBUTE DATA GENERATION METHOD, AND PROGRAM

(71) Applicant: NEC Corporation, Tokyo (JP)

(72) Inventors: Chenhui HUANG, Tokyo (JP); Kensuke WAGATA, Tokyo (JP); Fumiyuki NIHEY, Tokyo (JP)

(73) Assignee: NEC Corporation, Tokyo (JP)

(21) Appl. No.: 19/332,472

(22) Filed: Sep. 18, 2025

(30) Foreign Application Priority Data
Oct. 1, 2024 (JP) 2024-171957

(51) Int. Cl. G06F 18/2415; G16H 50/30

(52) U.S. Cl. CPC G06F 18/2415; G16H 50/30

(57) ABSTRACT

In order to generate insufficient attribute data by existing attribute data, a training device uses AI or machine learning models to train an attribute data generation device. An acquisition means acquires first and second attribute data other than the first attribute data. A first encoder converts the second attribute data into a stochastic latent variable. A second encoder projects the stochastic latent variable to a latent space, clusters projection points into clusters, and outputs centroids indicating centers of gravity of the clusters. A decoder reconstructs the second attribute data based on the projection points. An optimization means optimizes the first and second encoders, and the decoder based on relationships between the projection points and the centers of gravity and a relationship between the clusters. An analysis result of a health condition and a disease risk using the attribute data is used for supporting a decision making regarding a subject's activity.

### INCORPORATION BY REFERENCE

This application is based upon and claims the benefit of priority from Japanese Patent Application 2024-171957, filed on Oct. 1, 2024, the disclosure of which is incorporated herein in its entirety by reference.

### TECHNICAL FIELD

The present disclosure relates to generation of attribute data.

### BACKGROUND ART

A disease risk estimation technology using a machine learning model is known. For instance, Patent Document 1 describes a method of classifying data related to health into a group at high risk of disease and a group at high low of disease and evaluating a disease risk.

Patent Document 1: Japanese Patent Application Laid-Open under No. 2022-182943

### SUMMARY

In recent years, large-scale and annual health data can be acquired by a periodic medical examination or the like. However, in the health data obtained by the periodic medical examination or the like, variation of attributes of a subject is limited, and the attributes of data that can be acquired are biased. In order to perform prediction of a future health condition and estimation of a disease risk with high accuracy, it is needed to generate health data related to an insufficient attribute.

One object of the present disclosure is to provide an attribute data generation device capable of generating insufficient attribute data by using existing attribute data.

According to an example aspect of the present invention, there is provided a training device comprising:
at least one memory configured to store instructions; and
at least one processor configured to execute the instructions to:
acquire first attribute data and second attribute data other than the first attribute data;
convert, a first encoder, the second attribute data into a stochastic latent variable;
project, by a second encoder, the stochastic latent variable to a latent space according to a category of the first attribute data, clusters obtained projection points into a plurality of clusters, and outputs centroids indicating centers of gravity of the plurality of clusters;
reconstruct, by a decoder, the second attribute data based on the projection points in the latent space; and
optimize the first encoder, the second encoder, and the decoder based on relationships between the projection points in the latent space and the centers of gravity of the clusters and a mutual relationship between the plurality of clusters.

According to another example aspect of the present invention, there is provided a training method executed by a computer, the training method comprising: acquiring first attribute data and second attribute data other than the first attribute data;
converting, by using a first encoder, the second attribute data into a stochastic latent variable;
projecting, by using a second encoder, the stochastic latent variable to a latent space according to a category of the first attribute data, clustering obtained projection points into a plurality of clusters, and outputting centroids indicating centers of gravity of the plurality of clusters;
reconstructing, by using a decoder, the second attribute data based on the projection points in the latent space; and
optimizing the first encoder, the second encoder, and the decoder based on relationships between the projection points in the latent space and the centers of gravity of the clusters and a mutual relationship between the plurality of clusters.

According to still another example aspect of the present invention, there is provided a program for causing a computer to execute processing comprising:
acquiring first attribute data and second attribute data other than the first attribute data;
converting, by using a first encoder, the second attribute data into a stochastic latent variable;
projecting, by using a second encoder, the stochastic latent variable to a latent space according to a category of the first attribute data, clustering obtained projection points into a plurality of clusters, and outputting centroids indicating centers of gravity of the plurality of clusters;
reconstructing, by using a decoder, the second attribute data based on the projection points in the latent space; and
optimizing the first encoder, the second encoder, and the decoder based on relationships between the projection points in the latent space and the centers of gravity of the clusters and a mutual relationship between the plurality of clusters.

According to a further example aspect of the present invention, there is provided an attribute data generation device comprising:
at least one memory configured to store instructions; and
at least one processor configured to execute the instructions to:
acquire a category of a first attribute;
determine determining a projection point that belongs to a cluster corresponding to the category of the first attribute in a latent space obtained by clustering projection points obtained by projecting attribute data into a plurality of clusters; and
generate, by a decoder, second attribute data based on the projection point in the latent space.

According to a still further example aspect of the present invention, there is provided an attribute data generation method executed by a computer, the attribute data generation method comprising:
acquiring a category of a first attribute;
determining a projection point that belongs to a cluster corresponding to the category of the first attribute in a latent space obtained by clustering projection points obtained by projecting attribute data into a plurality of clusters; and
generating second attribute data based on the projection point in the latent space.

According to a yet still another example aspect of the present invention, there is provided a non-transitory computer-readable recording medium storing a program causing a computer to execute processing comprising:
acquiring a category of a first attribute;
determining a projection point that belongs to a cluster corresponding to the category of the first attribute in a latent space obtained by clustering projection points obtained by projecting attribute data into a plurality of clusters; and
generating second attribute data based on the projection point in the latent space.

According to the present disclosure, it is possible to generate insufficient attribute data by using existing attribute data.

### BRIEF DESCRIPTION OF THE DRAWINGS

FIG. 1 illustrates an overall configuration of an attribute data generation device according to the present disclosure;

FIG. 2 is a block diagram illustrating a hardware configuration of the attribute data generation device;

FIG. 3 is a block diagram illustrating a functional configuration of a training device;

FIG. 4 schematically illustrates a latent space;

FIG. 5 is a flowchart of training processing;

FIG. 6 is a block diagram illustrating a functional configuration of the attribute data generation device;

FIGS. 7A and 7B schematically illustrate the latent space;

FIG. 8 is a flowchart of attribute data generation processing;

FIG. 9 is a block diagram illustrating a functional configuration of another attribute data generation device;

FIG. 10 is a block diagram illustrating a functional configuration of another attribute data generation device;

FIG. 11 schematically illustrates a latent space;

FIG. 12 is a flowchart of another attribute data generation processing;

FIG. 13 is a block diagram illustrating a functional configuration of another training device;

FIG. 14 is a flowchart of another training processing;

FIG. 15 is a block diagram illustrating a functional configuration of another attribute data generation device; and

FIG. 16 is a flowchart of another attribute data generation processing.

### EXAMPLE EMBODIMENTS

Hereinafter, preferred example embodiments of the present disclosure will be described with reference to the drawings.

### First Example Embodiment

### [Overall Configuration]

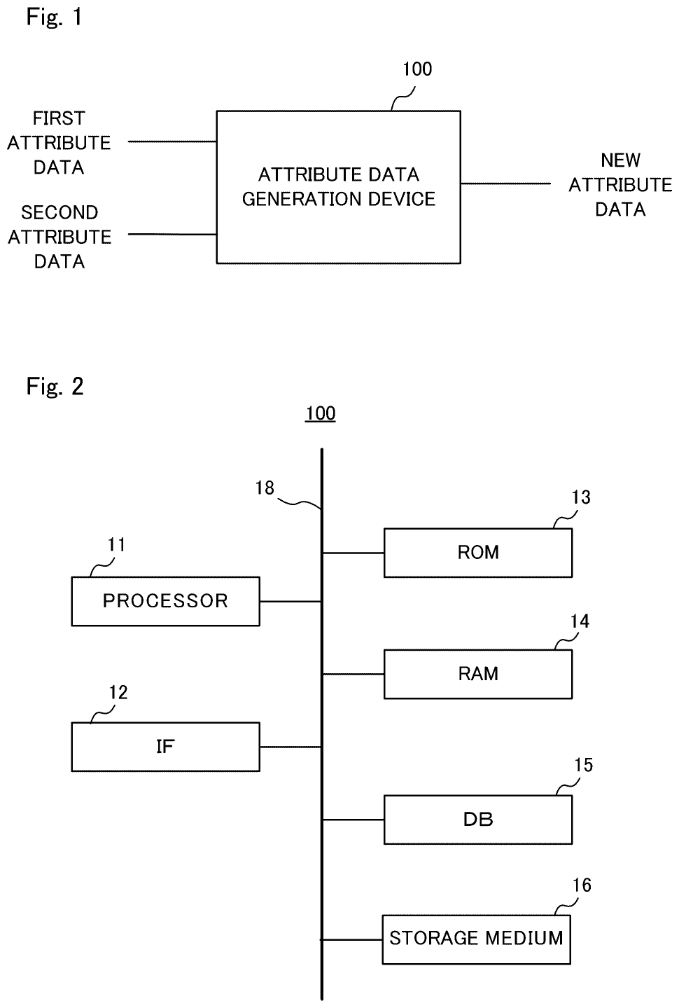

FIG. 1 illustrates an overall configuration of an attribute data generation device according to a first example embodiment of the present disclosure. An attribute data generation device 100 generates new attribute data based on existing attribute data related to health of a subject. The attribute data generation device 100 can be used to complement insufficient attribute data by using the existing attribute data.

Specifically, the attribute data generation device 100 receives input of first attribute data and second attribute data of the subject. The second attribute data include one or a plurality of pieces of attribute data which are other than the first attribute data. The first attribute data is attribute data related to a condition of new attribute data to be generated. The second attribute data is attribute data having the same attribute as that of the attribute data to be generated. Hereinafter, the new attribute data generated by the attribute data generation device 100 is also referred to as “object attribute data”.

In general, a periodic medical examination is aimed at prevention of lifestyle diseases and the like, and data of young people tends to be small. For instance, it is assumed that there are an insufficient number of pieces of blood pressure data of individuals in their 20s in health data collected by the periodic medical examination. In this case, the attribute data generation device 100 can generate the blood pressure data of the subjects in their twenties by using blood pressure data of all age groups collected by the periodic medical examination. In this case, the attribute data generation device 100 generates the object attribute data of the “blood pressure” by using the “age” as the first attribute data corresponding to the condition of the object attribute data to be generated and the “blood pressure” as the second attribute data. In this manner, the attribute data generation device 100 can generate the object attribute data corresponding to the insufficient condition by receiving the input of the first attribute data and the second attribute data.

The attribute data generation device 100 generates and outputs the attribute data of the subject by using an attribute data generation model, based on the first attribute data and the second attribute data. The attribute data generation model is an artificial intelligence (AI) or machine learning model trained by a training phase to be described later. The attribute data generation device 100 of the present disclosure can generate attribute data under an optional condition by using a probability distribution feature of each piece of the attribute data.

The attribute data generation device 100 can be suitably applied to a medical or healthcare field. For instance, the attribute data generation device 100 can be used to complement insufficient health data in a case where a risk of a lifestyle disease is estimated based on the health data obtained in the periodic medical examination. In addition, the attribute data generation device 100 can also be used to predict future health data based on current health data of the subject.

### [Hardware Configuration]

FIG. 2 is a block diagram illustrating a hardware configuration of the attribute data generation device 100. As illustrated in the drawing, the attribute data generation device 100 includes a processor 11, an interface (IF) 12, a read only memory (ROM) 13, a random access memory (RAM) 14, a database (DB) 15, and a storage medium 16. The components are connected to each other via, for instance, a bus 18.

The processor 11 is a computer such as a central processing unit (CPU), and controls the entire attribute data generation device 100 by executing a program prepared in advance. Specifically, as the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

Also, the processor 11 loads a program stored in the ROM 13 or the storage medium 16 into the RAM 14, and executes each type of processing coded in the program. The processor 11 functions as a part or all of the attribute data generation device 100. Specifically, the processor 11 executes training processing and attribute data generation processing to be described later.

The IF 12 transmits and receives data to and from an external device. Specifically, in the training phase, the attribute data generation device 100 receives existing attribute data obtained by the periodic medical examination or the like as training data via the IF 12. In a generation phase, that is, at the time of generation of attribute data, via the IF 12, the attribute data generation device 100 receives original attribute data, generates new attribute data (that is, object attribute data), and outputs the new attribute data to an external device.

The ROM 13 stores various programs executed by the processor 11. The RAM 14 is used as a working memory during execution of various types of processing by the processor 11.

The DB 15 stores various algorithms, data, a machine learning model, and the like used in a case where the attribute data generation device 100 executes the training processing and the attribute data generation processing to be described later.

The storage medium 16 is a non-volatile and non-transitory storage medium such as a disk-shaped recording medium or a semiconductor memory. The storage medium 16 may be attachable to and detachable from the attribute data generation device 100. The storage medium 16 records various programs executed by the processor 11.

In addition to the above, the attribute data generation device 100 may include a display device such as a liquid crystal display and an input device such as a keyboard and a mouse. The display device and the input device are used by, for instance, an operator of the attribute data generation device 100.

### [Training Phase]

Next, the training phase of the attribute data generation model will be described.

### (Training Device)

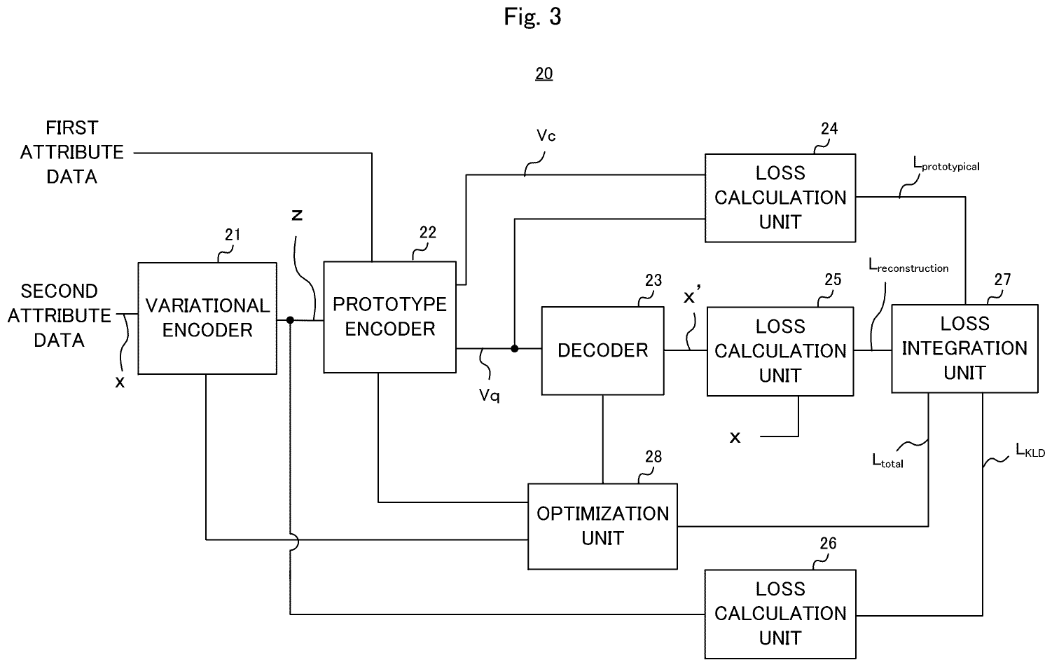

As described above, the attribute data generation device 100 generates the attribute data by using the trained attribute data generation model. FIG. 3 is a block diagram illustrating a functional configuration of a training device 20 of the attribute data generation model. The training device 20 trains the attribute data generation model by prototype training. As illustrated in the drawing, the training device 20 includes a variational encoder 21, a prototype encoder 22, a decoder 23, loss calculation units 24, 25, and 26, a loss integration unit 27, and an optimization unit 28.

The attribute data generation model basically is formed by combining the variational encoder 21, the prototype encoder 22, and the decoder 23. Specifically, the variational encoder 21, the prototype encoder 22, and the decoder 23 are configured by a neural network. In the training phase, the training device 20 generates the trained attribute data generation model by optimizing the neural network by using the training data.

As the training data, attribute data related to health of a plurality of persons are prepared. Specifically, the training data include, for instance, at least one of an age, a height, a weight, a gender, a body mass index (BMI), a blood pressure, a blood glucose level, presence or absence and amount of smoking, and presence or absence and amount of drinking.

In FIG. 3, first, the first attribute data and the second attribute data are input to the training device 20. The first attribute data is data that specifies an attribute of a prototype in the prototype training as a condition. In the following description, as an example, the first attribute data is set as the “age”. The second attribute data is attribute data other than the first attribute data, that is, one or a plurality of pieces of attribute data which are other than the age. Note that the second attribute data include the object attribute data generated by the attribute data generation device 100. That is, in a case where data of the attribute “blood pressure” is generated by using the attribute data generation device 100, the second attribute data include the data of the “blood pressure”.

First, second attribute data x is input to the variational encoder 21. The variational encoder 21 projects the input attribute data x to a stochastic latent space. The stochastic latent space is a low-dimensional latent space to which high-dimensional input data is mapped, and latent variables in the latent space follow a Gaussian distribution. That is, the variational encoder 21 converts the attribute data x into a latent variable z in the stochastic latent space, and outputs the latent variable z to the prototype encoder 22 and the loss calculation unit 26. The latent variable in the stochastic latent space is also referred to as a “stochastic latent variable”.

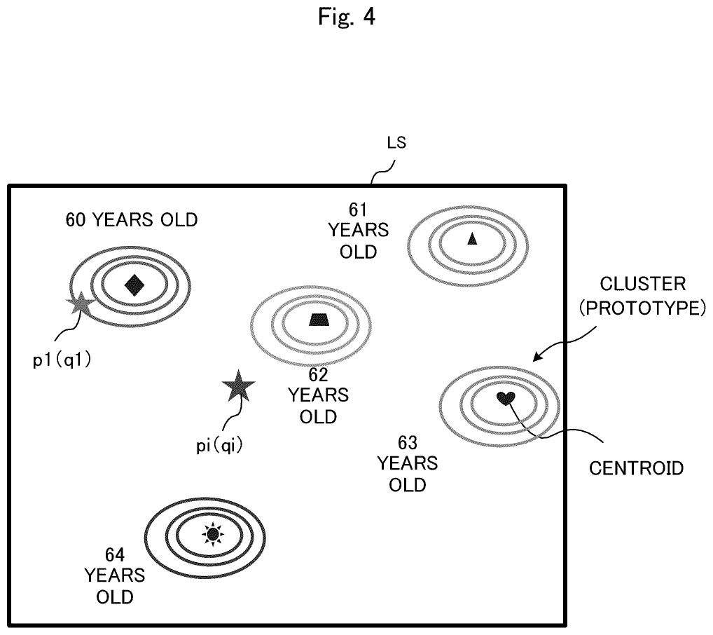

The prototype encoder 22 receives input of the first attribute data and also receives input of the latent variable z from the variational encoder 21. The prototype encoder 22 performs the prototype training by using the attribute specified by the first attribute data. Specifically, the prototype encoder 22 projects the input latent variable z to a latent space. FIG. 4 schematically illustrates a latent space LS used by the prototype encoder 22. Hereinafter, in order to distinguish from the stochastic latent space used by the variational encoder 21, the latent space LS used by the prototype encoder 22 may be referred to as a “prototype latent space” for convenience. The “latent space” is an abstract space for expressing information included in original data in fewer dimensions, and in the latent space, essential features and patterns of the data are expressed in the fewer dimensions. “Projects . . . to a latent space” refers to converting the original data into points on the latent space, which is also referred to as “maps . . . to a latent space”. Hereinafter, each point on a latent space obtained by projecting certain data to the latent space is also referred to as a “projection point”.

The prototype encoder 22 projects the second attribute data of the plurality of persons included in the training data to the latent space LS. As a result, a large number of the projection points are mapped onto the latent space LS. In FIG. 4, a position of the projection point in the latent space LS is denoted by “p”, and a feature representation CORRESPONDING to the position (also referred to as a “latent vector”, a “feature vector”, or simply a “vector” or the like) is denoted by “q”. In the example of FIG. 4, it is indicated that certain attribute data d1 is projected to a projection point p1 and a feature representation corresponding to the projection point p1 is q1. Similarly, it is indicated that certain attribute data di is projected to a projection point pi and a feature representation corresponding to the projection point pi is qi.

The prototype encoder 22 projects a plurality of pieces of second attribute data to the latent space LS according to the first attribute data (that is, the age), and clusters the obtained projection points. Specifically, the prototype encoder 22 clusters the projection points for each category of the age that is the first attribute data, and generates a cluster for each category of the age. The category of the age can be optionally set, and may be, for instance, a category for every one year of age, or a category for every five years of age. In the example of FIG. 4, the category of the age is set for every one year of age, and the variational encoder 21 generates clusters “60 years old”, “61 years old”, . . . for each age. These clusters are also referred to as “prototypes”, and a center of gravity of each cluster (prototype) is referred to as a “centroid”. In this manner, the prototype encoder 22 generates the cluster according to the category of the age based on the age input as the first attribute data.

After clustering the plurality of projection points, the prototype encoder 22 outputs, for each cluster, a feature representation of the center of gravity (hereinafter referred to as a ‘centroid vector’), denoted as Vc. The centroid vector Ve is represented by the following expression.

$$Vc = [ μ 1 , … , μ i , … , μ C ] \qquad ( 1 )$$

The centroid vector of each cluster is indicated by “μ”, and the number of clusters is indicated by “C”.

The prototype encoder 22 also outputs a feature representation (hereinafter referred to as a “projection point vector”) Vq of each projection point to the decoder 23 and the loss calculation unit 24. The projection point vector Vq is represented as follows. The number of projection points is indicated by “N”.

$$Vq = [ q 1 , … , qi , … , qN ] \qquad ( 2 )$$

The decoder 23 generates attribute data x′ based on the projection point vector Vq which is input. In other words, the decoder 23 generates the attribute data x′ obtained by reconstructing the input second attribute data x based on the projection point vector Vq, and outputs the attribute data x′ to the loss calculation unit 25.

The loss calculation unit 24 calculates a first loss L_{prototypical} by the following expression (3) by using the centroid vector Vc and projection point vector Vq which have been input, and outputs the first loss L_{prototypical} to the loss integration unit 27.

$$\begin{matrix}[ Math 1 ] & \\ {ℒ}_{prototypical} = {\underbrace{- \frac{1}{N} {\sum }_{i = 1}^{N} log ( \frac{exp ( - d ( {q}_{i} , {μ}_{i} ) )}{{\sum }_{j = 1}^{C} exp ( - d ( {q}_{i} , {μ}_{i} ) )} )}}_{First term} + {\underbrace{λ {\sum }_{j = 1}^{C} {\sum }_{k = 1 , k / j}^{C} \frac{1}{d ( {μ}_{j} , {μ}_{k} )}}}_{Second term} & ( 3 )\end{matrix}$$

In the expression (3), a function d (q, μ) indicates a distance between the projection point vector q and the centroid vector μ. Therefore, a denominator in parentheses in a first term of the expression (3) indicates a sum of distances between a certain projection point and centroids of clusters. A numerator in the parentheses in the first term indicates a distance between the projection point and a centroid of a cluster to which the projection point belongs. Therefore, the closer a projection point belonging to a cluster is to the centroid of that cluster, the smaller the value of the first term becomes. On the other hand, a second term of the expression (3) indicates a sum of reciprocals of distances between the individual centroids. Therefore, the farther apart individual centroids are, the smaller the value of the second term becomes. Therefore, the first loss L_{prototypical} decreases as a projection point belonging to a certain cluster is closer to a centroid of the cluster, and decreases as the individual centroids are farther from each other. Therefore, by using the first loss L_{prototypical}, the training device 20 performs training in such a way that a projection point in a cluster is close to a centroid of the cluster and centroids of clusters are far from each other in the latent space.

The loss calculation unit 25 calculates a reconstruction loss between the attribute data x′ reconstructed by the decoder 23 and the attribute data x input to the variational encoder 21 as a second loss L_{reconstruction}, and outputs the second loss L_{reconstruction} to the loss integration unit 27. As the reconstruction loss L_{reconstruction}, a square error or a cross entropy can be used.

The loss calculation unit 26 calculates Kullback-Leibler (KL) divergence between the latent variable z output from the variational encoder 21 and the Gaussian distribution as a third loss L_{KLD}, and outputs the third loss L_{KLD} to the loss integration unit 27. The KL divergence indicates similarity between two probability distributions. The third loss L_{KLD} is used to approximate the latent variable z output from the variational encoder 21 to the Gaussian distribution.

The loss integration unit 27 calculates a weighted sum of the first loss L_{prototypical}, the second loss L_{reconstruction}, and the third loss L_{KLD} by the following the expression (4), and outputs the weighted sum to the optimization unit 28 as a total loss Lₜₒₜₐₗ.

$$\begin{matrix}[ Math 2 ] & \\ {L}_{total} = {L}_{reconstruction} + α {L}_{prototypical} + β {L}_{KLD} & ( 4 )\end{matrix}$$

Note that ‘α’ and ‘β’ represent the weights used in a case of adding the first to third losses with weighted summation.

The optimization unit 28 optimizes the variational encoder 21, the prototype encoder 22, and the decoder 23 based on the total loss Lₜₒₜₐₗ. Specifically, the optimization unit 28 optimizes parameters of the neural network constituting the variational encoder 21, the prototype encoder 22, and the decoder 23 in such a way that the total loss Lₜₒₜₐₗ becomes small. Here, as described above, since the total loss Lₜₒₜₐₗ is the weighted sum of the first to third losses, the optimization unit 28 performs the optimization in such a way that, in the latent space, (A) a projection point in a cluster is close to a centroid of the cluster, and centroids of clusters are far from each other, (B) the reconstructed attribute data x′ is close to the original attribute data x, and (C) the latent variable output from the variational encoder 21 approaches the Gaussian distribution.

In this manner, the training device 20 generates the attribute data generation model for reconstructing the second attribute data by using the input first attribute data as the condition.

### (Training Processing)

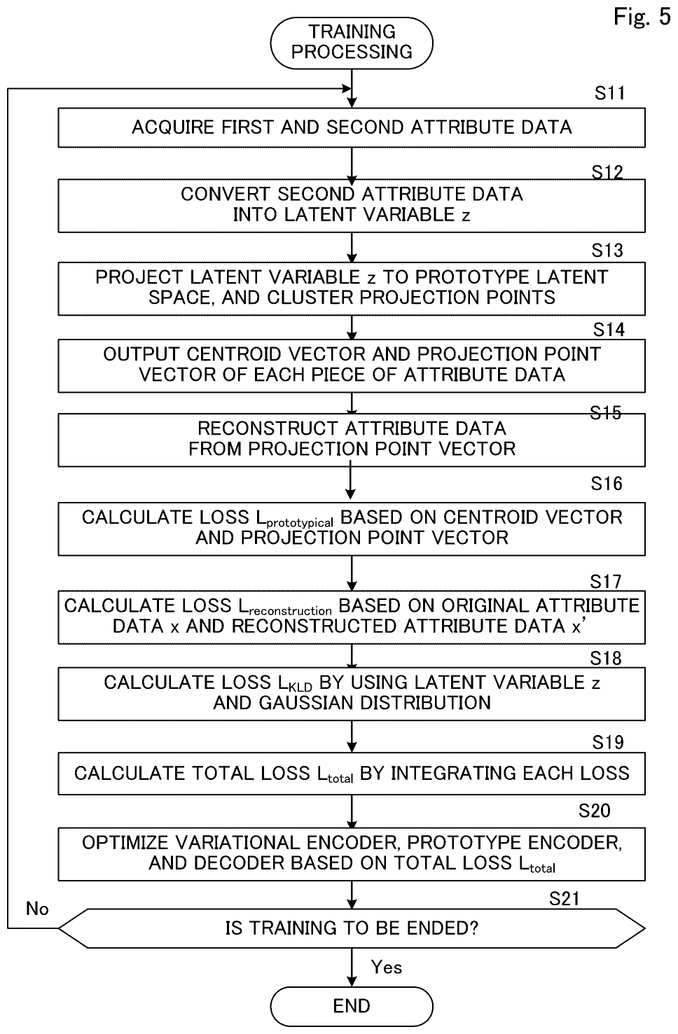

Next, the training processing executed by the above training device 20 will be described. FIG. 5 is a flowchart of the training processing. This training processing is achieved by the processor 11 illustrated in FIG. 2 executing a program prepared in advance and operating as the components illustrated in FIG. 3.

First, the training device 20 acquires the first attribute data and the second attribute data (step S11). Next, the variational encoder 21 converts the second attribute data into the latent variable z in the stochastic latent space (step S12). Next, the prototype encoder 22 projects the latent variable z to the prototype latent space LS, and clusters the obtained projection points (step S13). Next, the prototype encoder 22 outputs the centroid vector of each cluster and the projection point vector of each projection point (step S14). Next, the decoder 23 reconstructs the second attribute data based on the projection point vector, and generates the attribute data x′ (step S15).

Next, the loss calculation unit 24 calculates the first loss L_{prototypical} based on the centroid vector and the projection point vector of each projection point (step S16). The loss calculation unit 25 also calculates the second loss L_{reconstruction} based on the original attribute data x and the reconstructed attribute data x′ (step S17). Also, the loss calculation unit 26 calculates the third loss L_{KLD} by using the latent variable z and the Gaussian distribution (step S18). Note that steps S16 to S18 may be performed in any order or may be performed simultaneously.

Next, the loss integration unit 27 calculates the total loss Lₜₒₜₐₗ by integrating the first to third losses (step S19). Next, the optimization unit 28 optimizes the variational encoder 21, the prototype encoder 22, and the decoder 23 based on the total loss Lₜₒₜₐₗ (step S20).

Next, the training device 20 determines whether a predetermined training end condition is satisfied (step S21). The training end condition may be one of the following: a predetermined number of pieces of the attribute data prepared as the training data are used; the total loss becomes equal to or less than a predetermined value; or the total loss has converged. In a case where the training end condition is not satisfied (step S21: No), the training processing returns to step S11. On the other hand, in a case where the training end condition is satisfied (step S21: Yes), the training processing is terminated.

### [Generation Phase]

Next, the generation phase by the attribute data generation device will be described. In the generation phase, the attribute data generation device 100 generates attribute data related to health of a certain subject by using the attribute data of the subject.

### First Example

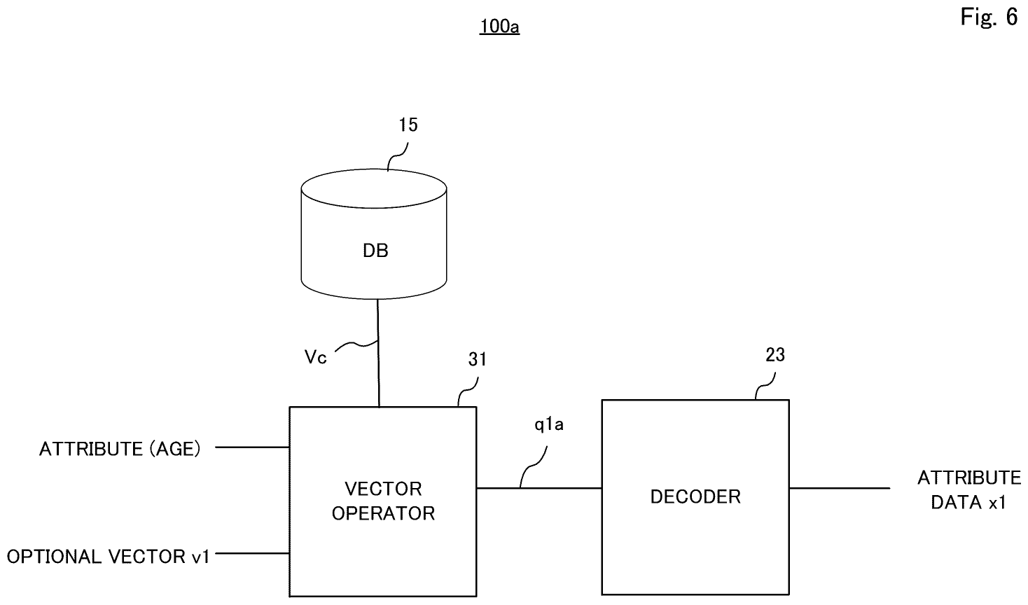

FIG. 6 is a block diagram illustrating a functional configuration of an attribute data generation device according to a first example. An attribute data generation device 100a includes a vector operator 31 and the decoder 23 optimized in the training phase.

The attribute data generation device 100a receives input of a first attribute and an optional vector. The first attribute corresponds to a condition in generation of the attribute data. In the following description, the first attribute is set as the “age”.

At the end of the training phase, the centroid vector Vc in the latent space LS used by the prototype encoder 22 is stored in a storage unit such as the DB 15. Specifically, for the latent space LS illustrated in FIG. 4, the clusters are generated for the age categories of 60 years old to 63 years old, and the centroid vector Vc corresponding to each age category is stored in the DB 15.

The vector operator 31 acquires the centroid vector Vc corresponding to the input first attribute (age) from the DB 15. The vector operator 31 then generates a projection point vector in the prototype latent space by using the centroid vector Vc and an optional vector v1 which is input. The optional vector v1 is a vector having the same number of dimensions as that of the centroid vector Vc.

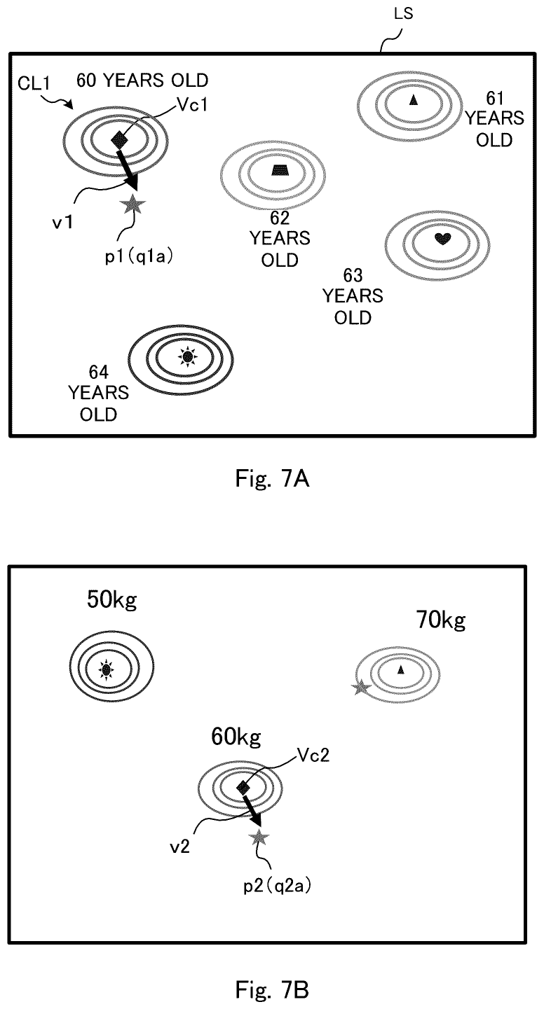

FIG. 7A is a conceptual diagram of the prototype latent space in the first example. Now, it is assumed that the age category “60 years old” is input as the first attribute. The vector operator 31 acquires a centroid vector Vel of a cluster CL1 corresponding to the 60 years old with reference to the DB 15, and generates a projection point vector q1a corresponding to the projection point p1 by using the centroid vector Vc1 and the optional vector v1. The vector operator 31 then outputs the projection point vector q1a to the decoder 23. The decoder 23 generates attribute data x1 corresponding to the optional vector v1 based on the projection point vector q1a.

For instance, in a case where training is performed by using the second attribute data including the “blood pressure” in the training phase, the attribute data generation device 100a can generate the attribute data x1 of the “blood pressure of 60 years old” corresponding to the optional vector v1. Moreover, in a case where the age category “62 years old” is input as the first attribute, the attribute data generation device 100a can generate the attribute data x1 of the “blood pressure of 62 years old” corresponding to the optional vector v1. On the other hand, in a case where the age category “60 years old” is input as the first attribute and a vector v1′ different from the vector v1 is input as the optional vector, the attribute data generation device 100a is capable of generating attribute data x1′ of the “blood pressure of 60 years old” corresponding to the vector v1′ different from the vector v1.

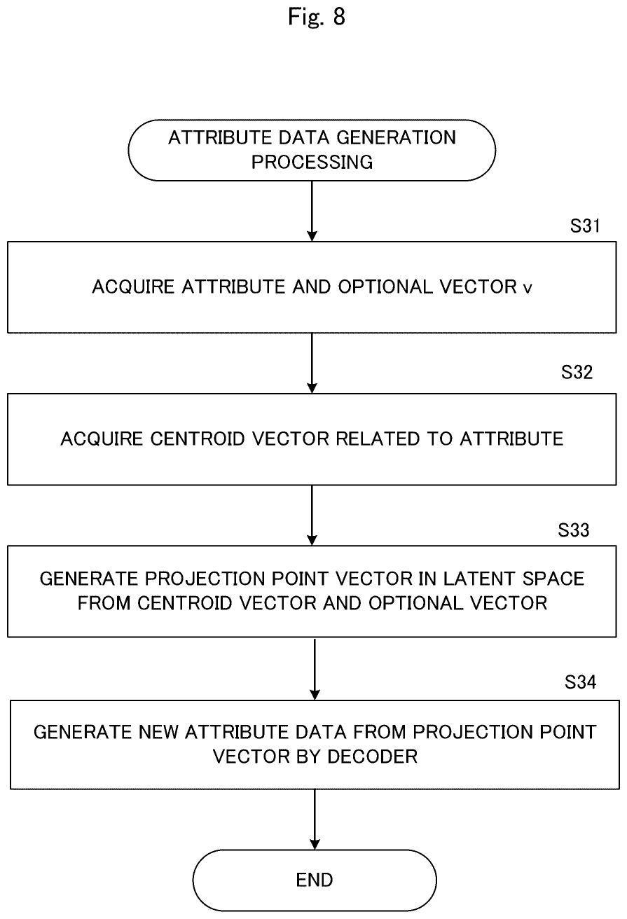

FIG. 8 is a flowchart of attribute data generation processing according to the first example. This processing is achieved by the processor 11 illustrated in FIG. 2 executing a program prepared in advance and operating as the attribute data generation device 100a illustrated in FIG. 6.

First, the attribute data generation device 100a acquires the first attribute and the optional vector (step S31). Next, the vector operator 31 acquires the centroid vector corresponding to the first attribute from the DB 15 (step S32), and generates the projection point vector in the latent space LS from the centroid vector and the optional vector (step S33). Next, the decoder 23 generates the attribute data corresponding to the first attribute based on the projection point vector (step S34). After that, the attribute data generation processing is terminated.

Next, an attribute data generation device according to a modification of the first example will be described. The above attribute data generation device 100a of the first example generates the attribute data with the one attribute (age) as the condition. Instead, the attribute data may be generated by using a plurality of attributes as the conditions.

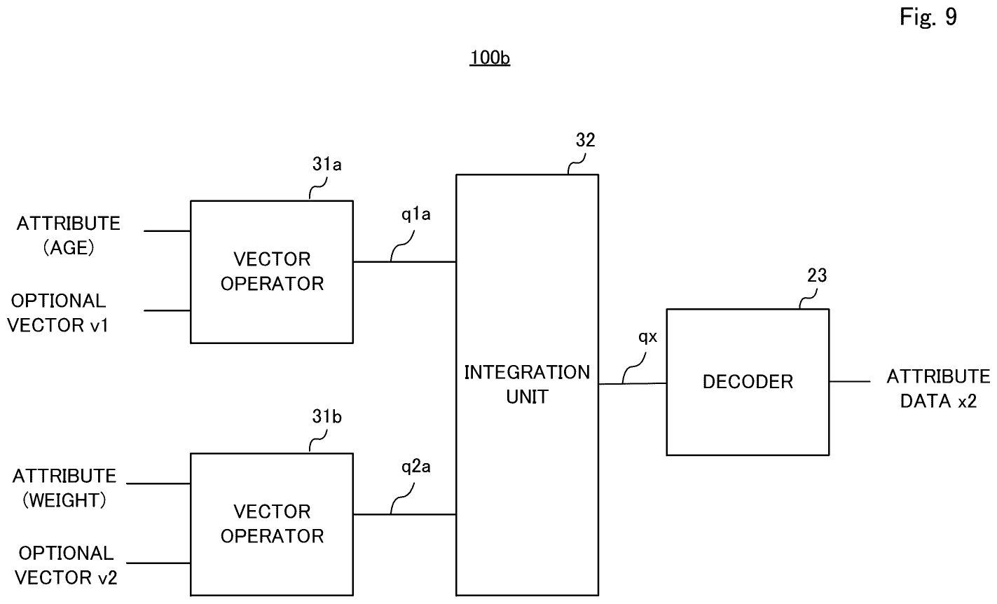

FIG. 9 is a block diagram illustrating a configuration of an attribute data generation device 100b that uses two attributes as conditions. As illustrated in the drawing, the attribute data generation device 100b includes two vector operators 31a and 31b, an integration unit 32, and the decoder 23. In this case, in the training phase, the latent space is subjected to the prototype training for the category of the age (60 years old, 61 years old, . . . ) as illustrated in FIG. 7A, and in addition, the latent space is subjected to the prototype training for a category of a weight (50 kg, 60 kg, . . . ) as illustrated in FIG. 7B. The centroid vector Ve of the prototype in each latent space is stored in the DB 15.

The vector operator 31a receives input of the category of the age as the attribute data, and further receives input of the optional vector v1. Now, it is assumed that “60 years old” is input as the category of the age. As illustrated in FIG. 7A, the vector operator 31a generates the projection point vector q1a at the projection point p1 by using the centroid vector Vel of the cluster of 60 years old and the optional vector v1, and outputs the projection point vector q1a to the integration unit 32.

The vector operator 31b receives input of the category of the weight as the attribute data, and further receives input of an optional vector v2. Now, it is assumed that “60 kg” is input as the category of the weight. As illustrated in FIG. 7B, the vector operator 31b generates a projection point vector q2a at a projection point p2 by using a centroid vector Vc2 of a cluster of 60 kg and the optional vector v2, and outputs the projection point vector q2a to the integration unit 32.

The integration unit 32 generates a vector qx by integrating the projection point vectors q1a and q2a, and outputs the vector qx to the decoder 23. Note that the integration unit 32 may integrate the vectors q1a and q2a by using, for instance, an attention mechanism, may use an average value of the vectors q1a and q2a as the vector qx, or may generate the vector qx by connecting the vectors q1a and q2a.

Based on the input vector qx, the decoder 23 generates attribute data x2 corresponding to the two input attributes, that is, “60 years old age, 60 kg weight”. In a case where it is assumed that training is performed by using the second attribute data including the “blood pressure” in the training phase, the attribute data generation device 100b can generate blood pressure data corresponding to “60 years old age, 60 kg weight”.

### Second Example

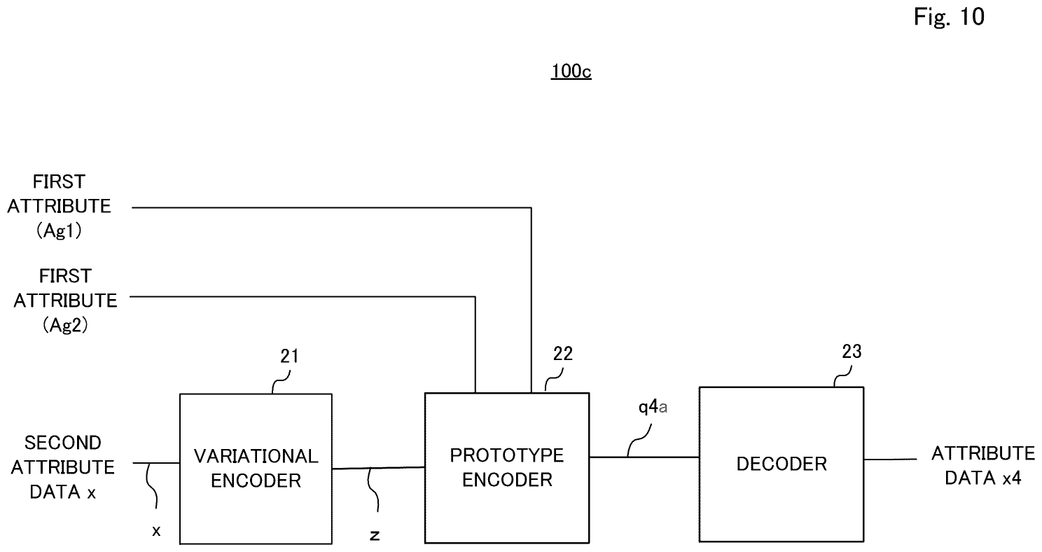

FIG. 10 is a block diagram illustrating a functional configuration of an attribute data generation device according to a second example. An attribute data generation device 100c includes the variational encoder 21, the prototype encoder 22, and the decoder 23. The variational encoder 21, the prototype encoder 22, and the decoder 23 are all optimized in the training phase.

The attribute data generation device 100c receives input of the first attribute and the second attribute data x. The first attribute relates to a condition in generation of the attribute data. In the following description, the first attribute is set as the “age”. In this example, a current age Ag1 and an age desired to be predicted (future age) Ag2 are input as the first attributes. As the age Ag2, the future age itself may be input, or the number of years (after N years) from the current age may be input. In the following example, it is assumed that “60 years old” is input as the current age Ag1 and “61 years old” is input as the future age Ag2. The second attribute data x is data of an attribute desired to be predicted, and it is assumed that the “blood pressure” is included in this example.

The variational encoder 21 converts the input attribute data x into the latent variable z in the stochastic latent space, and outputs the latent variable z to the prototype encoder 22. The first attributes Ag1 (60 years old) and Ag2 (61 years old) are input to the prototype encoder 22. Based on the first attributes Ag1 and Ag2 and the latent variable z input from the variational encoder 21, the prototype encoder 22 generates a projection point vector corresponding to the age of 61 years old desired to be predicted in the prototype latent space LS trained in the training phase.

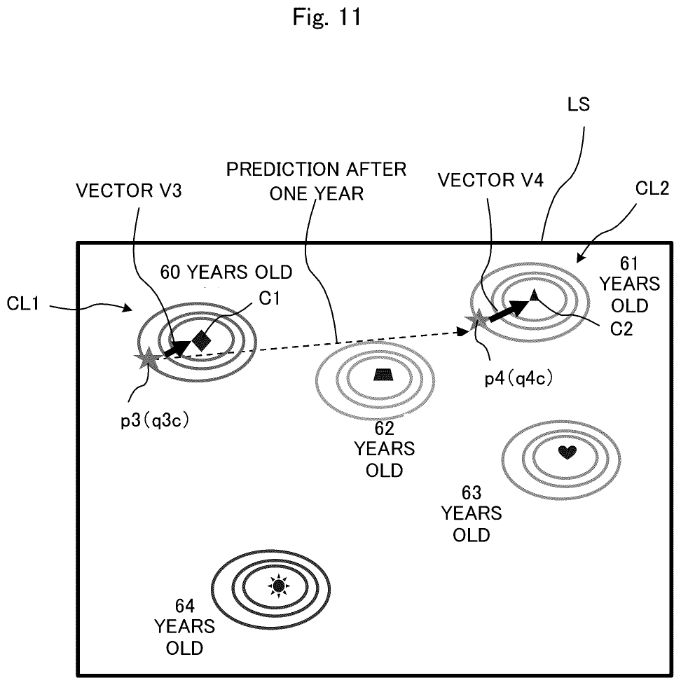

FIG. 11 schematically illustrates the latent space LS of the prototype encoder 22. The prototype encoder 22 first determines a projection point p3 of the latent variable z in the cluster of 60 years old in the latent space LS based on the attribute Ag1 (60 years old) and the latent variable z. Next, the prototype encoder 22 determines a projection point p4 corresponding to the future age Ag2, that is, 61 years old, based on the projection point p3 corresponding to the current age Ag1.

Specifically, the prototype encoder 22 moves the projection point p3 in the cluster CL1 of 60 years old corresponding to the current age Ag1 to a cluster CL2 of 61 years old corresponding to the future age Ag2, and sets the projection point p3 moved to the cluster CL2 as the projection point p4 corresponding to the future age Ag2. At this time, the variational encoder 21 generates the projection point p4 in such a way that a positional relationship between the projection point p3 and a centroid C1 in the cluster CL1 of 60 years old matches a positional relationship between the projection point p4 and a centroid C2 in the cluster CL2 of 61 years old after the movement. In other words, the prototype encoder 22 generates the projection point p4 in such a way that a vector V3 from the projection point p3 toward the centroid C1 in the cluster CL1 of 60 years old matches a vector V4 from the projection point p4 toward the centroid C2 in the cluster CL2 of 61 years old. As a result, the projection point p4 becomes a projection point indicating the feature representation in a case where the other attributes do not change and only the age changes to 61 years old for the subject. The prototype encoder 22 then outputs a projection point vector q4c of the determined projection point p4 to the decoder 23.

Based on the input projection point vector q4c, the decoder 23 generates the second attribute data at the age Ag2 desired to be predicted, that is, attribute data x4 corresponding to the “blood pressure of 61 years old”.

In this manner, according to the attribute data generation device 100c of the second example, for the first attribute “age”, the current attribute category “60 years old” and the attribute category desired to be predicted “61 years old” are specified, and the current attribute data x including the attribute “blood pressure” to be predicted is input, whereby the attribute data of the “blood pressure” in “61 years old” can be generated. In this case, the attribute data generation device 100c can output, for instance, a message such as “Blood pressure is predicted to be x4 after one year (61 years old).” to the subject.

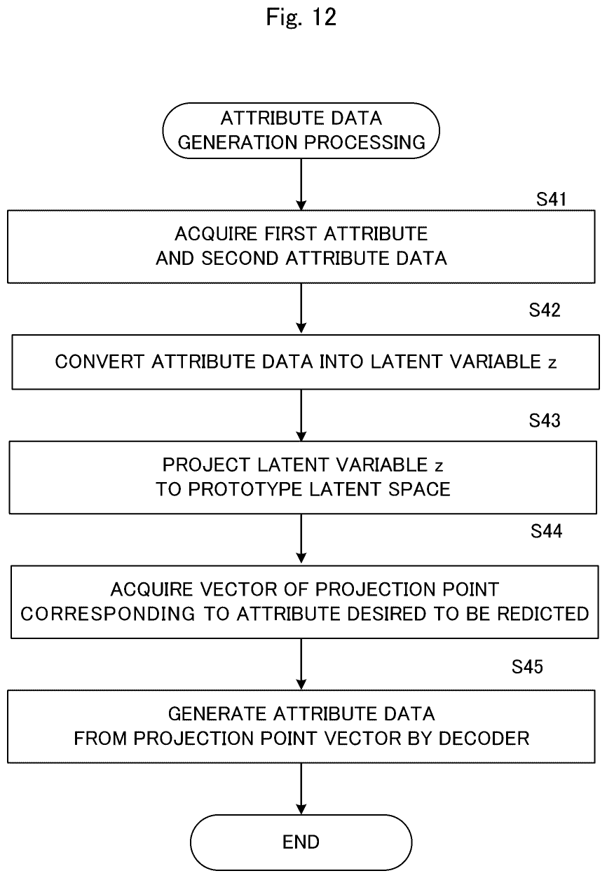

FIG. 12 is a flowchart of attribute data generation processing according to the second example. This processing is achieved by the processor 11 illustrated in FIG. 2 executing a program prepared in advance and operating as the attribute data generation device 100c illustrated in FIG. 10.

First, the attribute data generation device 100c acquires the first attributes Ag1 and Ag2 and the second attribute data x (step S41). Next, the variational encoder 21 converts the second attribute data x into the latent variable z in the stochastic latent space (step S42). Next, the prototype encoder 22 projects the latent variable z to the prototype latent space LS (step S43), moves the projection point from the cluster CL1 corresponding to the current attribute Ag1 to the cluster CL2 corresponding to the attribute Ag2 desired to be predicted, and acquires the projection point vector corresponding to the attribute Ag2 desired to be predicted (step S44). Next, the decoder 23 generates the attribute data corresponding to the second attribute based on the obtained projection point vector (step S45). After that, the attribute data generation processing then is terminated.

In the above example, the current attribute data x is used as the second attribute data (blood pressure). Instead, by using attribute data in an assumed state as the second attribute data, it is possible to predict how future attribute data changes in the case of the assumed state.

For instance, in the attribute data generation device 100c illustrated in FIG. 10, the current age Ag1 and the age Ag2 desired to be predicted are input as the first attributes (age). Moreover, a BMI is used as the second attribute, but the attribute data x is input, which include, instead of a current BMI, a BMI assumed to be lower than the current BMI, that is, the BMI lower than the actual BMI.

Also in this case, the attribute data generation device 100c operates in a manner similar to that described above. However, since the second attribute data x in a case where it is assumed that the BMI has lowered is input, the attribute data generation device 100c outputs the attribute data x4 after one year in that case. In this manner, the attribute data generation device 100c can predict the health data after one year in a case where it is assumed that the BMI has lowered.

### [Modification]

In the above first example embodiment, the age is used as the first attribute, but application of the present disclosure is not limited to this. The first attribute and the second attribute can be optionally set. For instance, when the weight, the BMI, or the like is used as the first attribute, prediction of other health data in a case where the weight or the BMI increases can be performed.

In the above first example embodiment, the attribute data generation device is applied to generation of attribute data related to health of a person, but application of the present disclosure is not limited to this. For instance, the present disclosure can also be applied to generation of attribute data detected and collected in inspection and diagnosis by a machine or a device.

### Second Example Embodiment

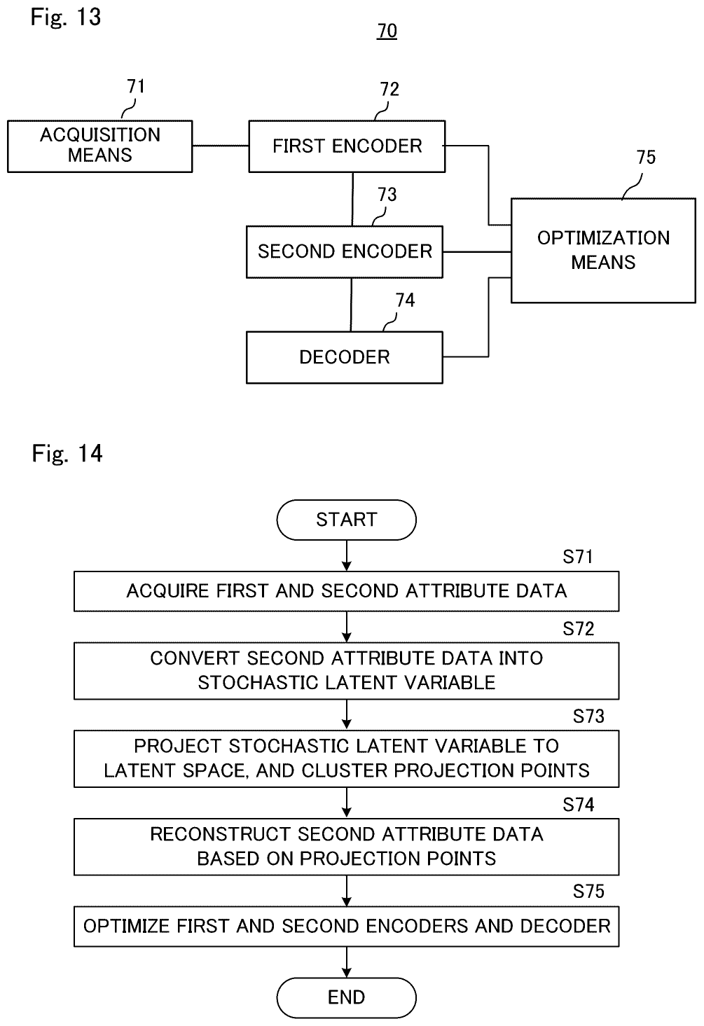

FIG. 13 is a block diagram illustrating a functional configuration of a training device according to a second example embodiment. A training device 70 includes an acquisition means 71, a first encoder 72, a second encoder 73, a decoder 74, and an optimization means 75.

FIG. 14 is a flowchart of processing by the training device 70. The acquisition means 71 acquires first attribute data and second attribute data other than the first attribute data (step S71). The first encoder 72 converts the second attribute data into a stochastic latent variable (step S72). The second encoder 73 projects the stochastic latent variable to a latent space according to a category of the first attribute data, clusters obtained projection points into a plurality of clusters, and outputs centroids indicating centers of gravity of the plurality of clusters (step S73). The decoder 74 reconstructs the second attribute data based on the projection points in the latent space (step S74). The optimization means 75 optimizes the first encoder, the second encoder, and the decoder based on relationships between the projection points in the latent space and the centers of gravity of the clusters and a mutual relationship between the plurality of clusters (step S75).

According to the training device 70 of the second example embodiment, it is possible to train an attribute data generation model capable of generating insufficient attribute data by using existing attribute data.

### Third Example Embodiment

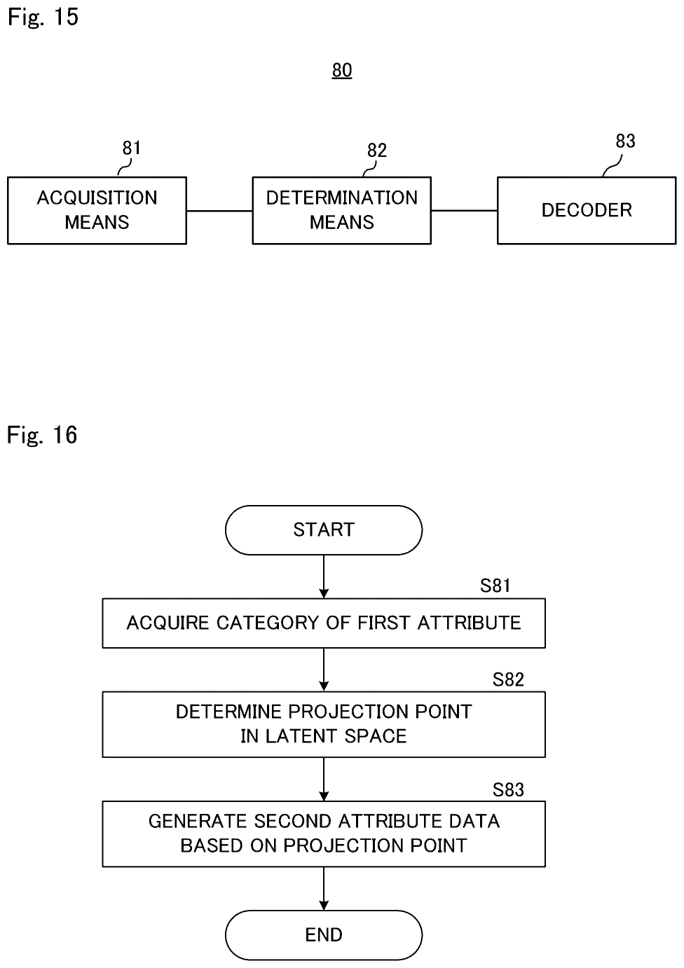

FIG. 15 is a block diagram illustrating a functional configuration of an attribute data generation device of a third example embodiment. An attribute data generation device 80 includes an acquisition means 81, a determination means 82, and a decoder 83.

FIG. 16 is a flowchart of processing by the attribute data generation device 80. The acquisition means 81 acquires a category of a first attribute (step S81). The determination means 82 determines a projection point belonging to a cluster corresponding to the category of the first attribute in a latent space obtained by clustering projection points obtained by projecting attribute data into a plurality of clusters (step S82). The decoder 83 generates second attribute data based on the projection point in the latent space (step S83).

According to the attribute data generation device 80 of the third example embodiment, it is possible to generate insufficient attribute data by using existing attribute data.

Some or all of the above example embodiments can also be described as the following Supplementary Notes, but are not limited to the following Supplementary Notes.

### (Supplementary Note 1)

A training device comprising:
an acquisition configured to acquire first attribute data and second attribute data other than the first attribute data;
a first encoder that converts the second attribute data into a stochastic latent variable;
a second encoder that projects the stochastic latent variable to a latent space according to a category of the first attribute data, clusters obtained projection points into a plurality of clusters, and outputs centroids indicating centers of gravity of the plurality of clusters;
a decoder that reconstructs the second attribute data based on the projection points in the latent space; and
an optimization configured to optimize the first encoder, the second encoder, and the decoder based on relationships between the projection points in the latent space and the centers of gravity of the clusters and a mutual relationship between the plurality of clusters.

### (Supplementary Note 2)

The training device according to supplementary note 1, wherein the first attribute data and the second attribute data are attribute data related to health.

### (Supplementary Note 3)

A training method executed by a computer, the training method comprising:
acquiring first attribute data and second attribute data other than the first attribute data;
converting, by using a first encoder, the second attribute data into a stochastic latent variable;
projecting, by using a second encoder, the stochastic latent variable to a latent space according to a category of the first attribute data, clustering obtained projection points into a plurality of clusters, and outputting centroids indicating centers of gravity of the plurality of clusters;
reconstructing, by using a decoder, the second attribute data based on the projection points in the latent space; and
optimizing the first encoder, the second encoder, and the decoder based on relationships between the projection points in the latent space and the centers of gravity of the clusters and a mutual relationship between the plurality of clusters.

### (Supplementary Note 4)

A program for causing a computer to execute processing comprising:
acquiring first attribute data and second attribute data other than the first attribute data;
converting, by using a first encoder, the second attribute data into a stochastic latent variable;
projecting, by using a second encoder, the stochastic latent variable to a latent space according to a category of the first attribute data, clustering obtained projection points into a plurality of clusters, and outputting centroids indicating centers of gravity of the plurality of clusters;
reconstructing, by using a decoder, the second attribute data based on the projection points in the latent space; and
optimizing the first encoder, the second encoder, and the decoder based on relationships between the projection points in the latent space and the centers of gravity of the clusters and a mutual relationship between the plurality of clusters.

### (Supplementary Note 5)

An attribute data generation device comprising:
acquisition configured to acquire a category of a first attribute;
determination configured to determine a projection point that belongs to a cluster corresponding to the category of the first attribute in a latent space obtained by clustering projection points obtained by projecting attribute data into a plurality of clusters; and
a decoder that generates second attribute data based on the projection point in the latent space.

### (Supplementary Note 6)

The attribute data generation device according to supplementary note 5, wherein the determination means acquires an optional vector, and determines the projection point based on a relationship between the optional vector in the latent space and a centroid of the cluster corresponding to the category of the first attribute.

### (Supplementary Note 7)

The attribute data generation device according to supplementary note 5, wherein
the acquisition means further acquires the second attribute data having an attribute other than the first attribute, and
the determination means includes:
a first encoder that converts the second attribute data into a stochastic latent variable; and
a second encoder that projects the stochastic latent variable to the latent space according to the category of the first attribute and determines a projection point of the second attribute data in the latent space.

### (Supplementary Note 8)

The attribute data generation device according to supplementary note 7, wherein
the category of the first attribute includes a current age and a future age of a subject, and
the determination means moves a projection point corresponding to the current age in the latent space to a position corresponding to the future age, and determines a projection point corresponding to the future age.
   (Supplementary note 9)

An attribute data generation method executed by a computer, the attribute data generation method comprising:
acquiring a category of a first attribute;
determining a projection point that belongs to a cluster corresponding to the category of the first attribute in a latent space obtained by clustering projection points obtained by projecting attribute data into a plurality of clusters; and
generating second attribute data based on the projection point in the latent space.
   (Supplementary note 10)

A program for causing a computer to execute processing comprising:
acquiring a category of a first attribute;
determining a projection point that belongs to a cluster corresponding to the category of the first attribute in a latent space obtained by clustering projection points obtained by projecting attribute data into a plurality of clusters; and
generating second attribute data based on the projection point in the latent space.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

11 Processor
20 Training device
21 Variational encoder
22 Prototype encoder
23 Decoder
24, 25, 26 Loss calculation unit
27 Loss integration unit
28 Optimization unit
100, 100a, 100b, 100c Attribute data generation device

1. A training device comprising:
at least one memory configured to store instructions; and
at least one processor configured to execute the instructions to:
acquire first attribute data and second attribute data other than the first attribute data;
convert, a first encoder, the second attribute data into a stochastic latent variable;
project, by a second encoder, the stochastic latent variable to a latent space according to a category of the first attribute data, clusters obtained projection points into a plurality of clusters, and outputs centroids indicating centers of gravity of the plurality of clusters;
reconstruct, by a decoder, the second attribute data based on the projection points in the latent space; and
optimize the first encoder, the second encoder, and the decoder based on relationships between the projection points in the latent space and the centers of gravity of the clusters and a mutual relationship between the plurality of clusters.

2. The training device according to claim 1, wherein the first attribute data and the second attribute data are attribute data related to health.

3. A training method executed by a computer, the training method comprising:
acquiring first attribute data and second attribute data other than the first attribute data;
converting, by using a first encoder, the second attribute data into a stochastic latent variable;
projecting, by using a second encoder, the stochastic latent variable to a latent space according to a category of the first attribute data, clustering obtained projection points into a plurality of clusters, and outputting centroids indicating centers of gravity of the plurality of clusters;
reconstructing, by using a decoder, the second attribute data based on the projection points in the latent space; and
optimizing the first encoder, the second encoder, and the decoder based on relationships between the projection points in the latent space and the centers of gravity of the clusters and a mutual relationship between the plurality of clusters.

4. A program for causing a computer to execute processing comprising:
acquiring first attribute data and second attribute data other than the first attribute data;
converting, by using a first encoder, the second attribute data into a stochastic latent variable;
projecting, by using a second encoder, the stochastic latent variable to a latent space according to a category of the first attribute data, clustering obtained projection points into a plurality of clusters, and outputting centroids indicating centers of gravity of the plurality of clusters;
reconstructing, by using a decoder, the second attribute data based on the projection points in the latent space; and
optimizing the first encoder, the second encoder, and the decoder based on relationships between the projection points in the latent space and the centers of gravity of the clusters and a mutual relationship between the plurality of clusters.

5. An attribute data generation device comprising:
at least one memory configured to store instructions; and
at least one processor configured to execute the instructions to:
acquire a category of a first attribute;
determine determining a projection point that belongs to a cluster corresponding to the category of the first attribute in a latent space obtained by clustering projection points obtained by projecting attribute data into a plurality of clusters; and
generate, by a decoder, second attribute data based on the projection point in the latent space.

6. The attribute data generation device according to claim 5, wherein the processor acquires an optional vector, and determines the projection point based on a relationship between the optional vector in the latent space and a centroid of the cluster corresponding to the category of the first attribute.

7. The attribute data generation device according to claim 5, wherein
the processor further acquires the second attribute data having an attribute other than the first attribute, and
in moving the projection point, the processor is configured to,
convert, by a first encoder, the second attribute data into a stochastic latent variable; and
project, by a second encoder, the stochastic latent variable to the latent space according to the category of the first attribute and determines a projection point of the second attribute data in the latent space.

8. The attribute data generation device according to claim 7, wherein
the category of the first attribute includes a current age and a future age of a subject, and
the processor moves a projection point corresponding to the current age in the latent space to a position corresponding to the future age, and determines a projection point corresponding to the future age.

9. An attribute data generation method executed by a computer, the attribute data generation method comprising:
acquiring a category of a first attribute;
determining a projection point that belongs to a cluster corresponding to the category of the first attribute in a latent space obtained by clustering projection points obtained by projecting attribute data into a plurality of clusters; and
generating second attribute data based on the projection point in the latent space.

10. A non-transitory computer-readable recording medium storing a program causing a computer to execute processing comprising:
acquiring a category of a first attribute;
determining a projection point that belongs to a cluster corresponding to the category of the first attribute in a latent space obtained by clustering projection points obtained by projecting attribute data into a plurality of clusters; and
generating second attribute data based on the projection point in the latent space.
